(19) United States
(12) Plant Patent Application Publication
(10) Pub. No.: US 2017/0280603 P1
(43) Pub. Date: Sep. 28, 2017

(54) Strawberry plant named 'Drisstrawfifty'

(50) ;

(71) Applicant: Driscoll's, Inc., Watsonville, CA (US)

(72) Inventors: Matthias D. Vitten, Aptos, CA (US); Raúl Fernández Sánchez, Huelva (ES); Carlos D. Fear, Aptos, CA (US); Katalin Pakozdi, Maidstone (GB)

(21) Appl. No.: 14/999,062

(22) Filed: Mar. 25, 2016

(51) Int. Cl. A01H 5/08

(52) U.S. Cl. PLT/208

(57) ABSTRACT

A new and distinct variety of strawberry plant named ‘DrisStrawFifty’ particularly characterized by having medium-sized, dark orange-red fruit, a globose semi-upright plant habit, and mid-season harvest maturity, is disclosed.

Genus and species: Fragaria×ananassa.

Variety denomination: ‘DrisStrawFifty’.

### BACKGROUND OF THE NEW PLANT

The present invention relates to a new and distinct strawberry variety designated ‘DrisStrawFifty’ and botanically known as Fragaria×ananassa. This new strawberry variety was discovered in Huelva, Spain in March 2010, and originated from a cross between the proprietary female parent ‘DrisStrawSixteen’ (U.S. Plant Pat. No. 22,247) and the proprietary male parent ‘RES 070-001’ (unpatented). A single plant was selected and asexually propagated via tissue culture and vegetative cuttings in Huelva, Spain in 2010.

‘DrisStrawFifty’ underwent further testing in Dofiana, Huelva, Spain from 2011-2015. The present invention has been found to retain its distinctive characteristics through successive asexual propagations via stolons and tissue culture.

Plant Breeder's Rights for this variety have not been applied for. ‘DrisStrawFifty’ has not been made publicly available or sold anywhere in the world more than one year prior to the filing date of this application.

### SUMMARY OF THE INVENTION

The following are the most outstanding and distinguishing characteristics of this new cultivar when grown under normal horticultural practices in Huelva, Spain.
1. Medium-sized, dark orange-red fruit;
2. Globose semi-upright plant habit; and
3. Mid-season harvest maturity.

### DESCRIPTION OF THE PHOTOGRAPHS

The accompanying color photographs show typical specimens of the new variety at various stages of development. The colors shown are as true as can be reasonably obtained by conventional photographic procedures. The photographs were taken from six-month-old plants.

FIG. 1 shows upper and lower surfaces of the leaves of the plant with three leaflets.

FIG. 2 shows both upper and lower surfaces of the flowers.

FIG. 3 shows the whole fruit.

FIG. 4 shows the fruit in longitudinal cross-section.

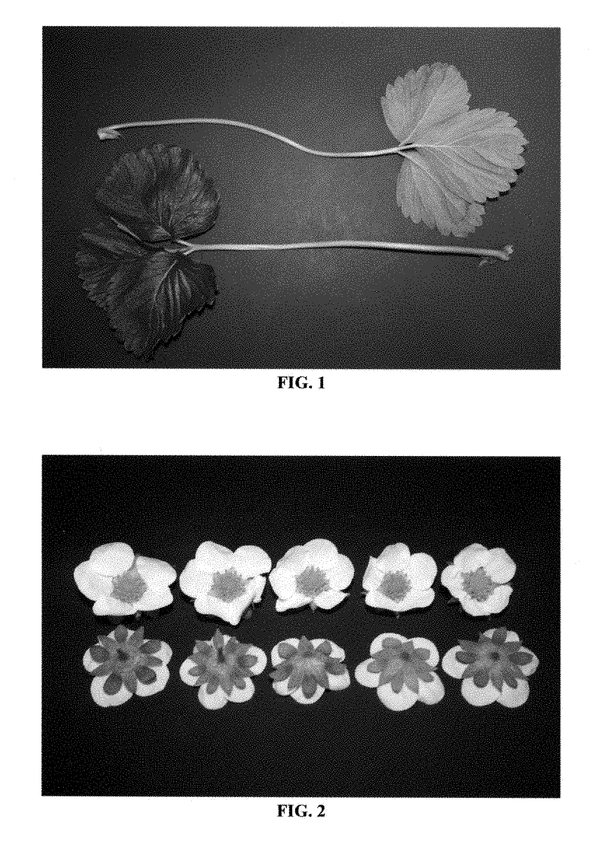

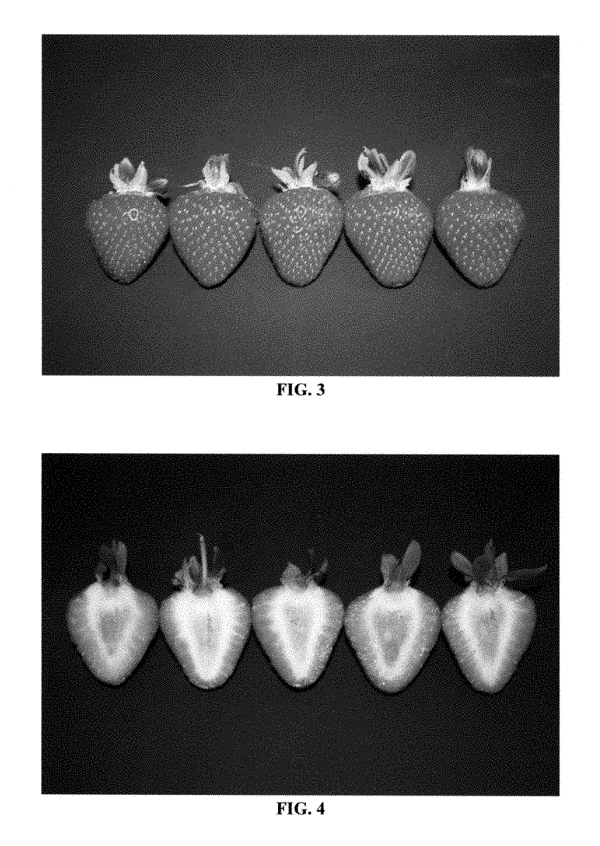

### DESCRIPTION OF THE NEW VARIETY

The following detailed descriptions set forth the distinctive characteristics of ‘DrisStrawFifty’. The data which define these characteristics is based on observations taken in Huelva, Spain from 2011 to 2015. This description is in accordance with UPOV terminology. Color designations, color descriptions, and other phenotypical descriptions may deviate from the stated values and descriptions depending upon variation in environmental, seasonal, climatic, and cultural conditions. ‘DrisStrawFifty’ has not been observed under all possible environmental conditions. The botanical description of ‘DrisStrawFifty’ was taken from six-month-old plants. Color references are primarily to the R.H.S. Colour Chart of The Royal Horticultural Society of London (R.H.S.) (2007 edition). Descriptive terminology follows the Plant Identification Terminology, An Illustrated Glossary, 2^{nd} edition by James G. Harris and Melinda Woolf Harris, unless where otherwise defined.

### DETAILED BOTANICAL DESCRIPTION OF THE PLANT

Classification:
   Species.—Fragaria×ananassa.
   Common name.—Strawberry.
   Denomination.—‘DrisStrawFifty’.
Parentage:
   Female parent.—The proprietary variety ‘DrisStrawSixteen’ (U.S. Plant Pat. No. 22,247).
   Male parent.—The proprietary variety ‘RES 070-001’ (unpatented).
Plant:
   Height.—22.5 cm.
   Diameter.—35.4 cm.
   Number of crowns/plant.—3.
   Habit.—Globose — semi-upright.
   Density of individual plant.—Medium.
   Vigor (health and hardiness of plant).—Medium.
Terminal leaflets:
   Size.—Medium. Length: 7.2 cm Width: 7.1 cm Length/width ratio: 1.0 (As long as broad).
   Number of teeth/terminal leaflet.—21.
   Shape of teeth.—Obtuse — serrate to crenate.
   Color.—Upper surface: RHS N137B (Dark green) Lower surface: RHS 146B (Medium yellow-green).
   Shape in cross section.—Concave.
   Blistering.—Absent or very weak.
   Glossiness.—Medium.
   Number of leaflets.—Three only.
   Shape.—Orbicular.
   Base shape.—Rounded.
   Apex descriptor.—Rounded.
   Margin.—Crenate.
   Margin profile.—Involute (margins rolled inwards).
   Variegation.—Absent.
Petiole:
   Length.—Medium; 15.9 cm.
   Diameter.—3.30 mm.
   Pubescence.—Medium.
   Pose of hairs.—Slightly upwards.
   Color.—RHS 144C (Medium yellow-green).
   Bract frequency.—0.
Petiolule:
   Length.—9.60 mm.
   Diameter.—1.70 mm.
   Color.—RHS 145A (Medium yellow-green).
Stipule:
   Length.—3.7 cm.
   Width.—9.60 mm.
   Pubescence.—Medium.
   Stipule anthocyanin coloration.—Medium; RHS 145B (Medium yellow-green).
Stolon:
   Number.—Medium.
   Average number of daughter plants per square foot.—3.5.
   Anthocyanin coloration.—Strong; RHS 166A (Dark greyed-orange).
   Diameter at bract.—4.20 mm.
   Thickness.—Thin.
   Density of pubescence.—Sparse.
Inflorescence:
   Position relative to foliage.—Above.
   Number of flowers per plant.—Few.
   Time of flowering (50% of plants at first flower).—Medium.
   Flower size.—Medium.
   Flower diameter.—40.40 mm.
   Petals.—Shape: Orbicular Apex: Rounded Base: Concavo-convex Margin: Entire Spacing: Overlapping Length: 16.00 mm Width: 15.60 mm Length/width ratio: 1.0 (As long as broad) Petal number per flower: 5 Color (upper surface): RHS NN155C (White).
   Calyx.—Diameter: 47.00 mm Diameter relative to corolla: Larger Inner calyx diameter relative to outer: Larger Insertion of calyx: Set above fruit — raised Pose of calyx segments: Spreading — outwards Size of calyx in relation to fruit: Slightly larger Adherence of calyx: Medium.
   Sepal.—Shape: Elliptical Apex: Truncate Margin: Entire Length: 20.90 mm Width: 10.70 mm Sepal number: 10.
   Receptacle color.—RHS 5B (Medium yellow).
   Stamen.—Present Anther color: RHS 153B (Medium yellow-green).
   Pedicel.—Attitude of hairs: Slightly upwards.
Fruiting truss:
   Length.—Medium; 22.8 cm.
   Diameter at base of truss.—3.10 mm.
   Number of berries per fruiting truss.—3.
   Attitude at first picking.—Semi-erect.
   Color at base of truss.—RHS 145C (Light yellow-green).
Fruit:
   Relative fruit size.—Medium.
   Length.—39.20 mm.
   Width.—34.70 mm.
   Length/width ratio.—1.1 (Longer than broad).
   Fruit hollow length.—17.90 mm.
   Fruit hollow width.—10.20 mm.
   Fruit hollow length/width ratio.—1.8.
   Fruit hollow center (cavity).—Medium.
   Fruit weight.—25.5 g.
   Predominant fruit shape.—Conical.
   Difference in shape between primary and secondary fruits.—Slight.
   Evenness of fruit surface.—Even or very slightly uneven.
   Fruit skin color.—RHS N34A (Dark orange-red).
   Evenness of fruit color.—Slightly uneven.
   Fruit glossiness.—Weak.
   Achenes.—Insertion of achenes: Above the surface Coloration (sunward side of berry): RHS 144B (Medium yellow-green) Coloration (shaded side of berry): RHS 144C (Medium yellow-green) Number per berry: 260 Weight (weight of achenes divided by total # seed): 0.00058 g Width of band without achenes: Medium.
   Firmness of flesh (when fully ripe).—Medium.
   Color of flesh (excluding core).—RHS 44A (Dark red).
   Color of core.—RHS 44C (Medium red).
   Evenness of flesh color.—Even.
   Distribution of flesh color.—Marginal and central.
   Sweetness.—Medium to strong.
   Acidity.—Weak.
   Texture when tasted.—Fine.
   Type of bearing.—Not everbearing — not remontant.
   Harvest interval.—Early February to mid-May.
   Harvest maturity.—Mid-season.
   Production.—698.0 grams per plant of marketable fruit.
Disease and pest resistance:
   Powdery mildew.—Moderately resistant.

### COMPARISON WITH PARENTAL AND COMMERCIAL VARIETIES

When ‘DrisStrawFifty’ is compared to the female parent ‘DrisStrawSixteen’ (U.S. Plant Pat. No. 22,247), ‘DrisStrawFifty’ produces globose—semi-upright plants with medium vigor, while the plants of ‘DrisStrawSixteen’ have a globose habit with strong vigor. Additionally, plants of ‘DrisStrawFifty’ produce a medium number of stolons with strong anthocyanin coloration, while ‘DrisStrawSixteen’ plants produce few stolons, with little or no anthocyanin coloration.

When ‘DrisStrawFifty’ is compared to the male parent ‘RES 070-001’ (unpatented), ‘DrisStrawFifty’ has slightly smaller fruit than ‘RES 070-001’.

‘DrisStrawFifty’ differs from the commercial variety ‘DrisStrawEight’ (U.S. Plant Pat. No. 20,735), in that ‘DrisStrawFifty’ has medium-sized fruit that is dark orange-red in color with weak glossiness, whereas ‘DrisStrawEight’ has large fruit that is medium red in color with medium glossiness. Additionally, plants of ‘DrisStrawFifty’ have a globose—semi-upright habit, whereas plants of ‘DrisStrawEight’ have a globose habit.

We claim:

1. A new and distinct variety of strawberry plant named ‘DrisStrawFifty’ substantially as illustrated and described herein.
